(19) United States
(12) Patent Application Publication
(10) Pub. No.: US 2016/0109432 A1
(43) Pub. Date: Apr. 21, 2016

(54) METHODS AND MATERIALS FOR ASSESSING PLURIPOTENCY OF STEM CELL POPULATIONS

(71) Applicant: Mayo Foundation for Medical Education and Research, Rochester, MN (US)

(72) Inventors: Timothy J. Nelson, Rochester, MN (US); Alyson J. Smith, Hamilton, MT (US); Frank J. Secreto, Rochester, MN (US)

(73) Assignee: MAYO FOUNDATION FOR MEDICAL EDUCATION AND RESEARCH, Rochester, MN (US)

(21) Appl. No.: 14/884,873

(22) Filed: Oct. 16, 2015

Related U.S. Application Data
(60) Provisional application No. 62/065,483, filed on Oct. 17, 2014.

(51) Int. Cl. G01N 33/50

(52) U.S. Cl. CPC G01N 33/5073; G01N 2510/00

(57) ABSTRACT

This document provides methods and materials for assessing the pluripotency of stem cell populations. For example, methods and materials for using a DNA damaging agent (e.g., etoposide) to assess the pluripotency of a population of stem cells (e.g., a population of human induced pluripotent stem cells) are provided.

### CROSS-REFERENCE TO RELATED APPLICATIONS

This application claims the benefit of U.S. Provisional Application Ser. No. 62/065,483, filed Oct. 17, 2014. The disclosure of the prior application is considered part of (and is incorporated by reference in) the disclosure of this application.

### BACKGROUND

1. Technical Field

This document relates to methods and materials involved in assessing pluripotency of stem cell populations. For example, this document relates to methods and materials for using a DNA damaging agent (e.g., etoposide) to assess the pluripotency of a population of pluripotent stem cells (e.g., a population of human induced pluripotent stem cells).

2. Background Information

Human induced pluripotent stem cells hold great promise as a regenerative platform for an array of clinical maladies. Production of large repositories of these cells at several institutions is underway.

### SUMMARY

This document provides methods and materials for assessing the pluripotency of stem cell populations. For example, this document provides methods and materials for using a DNA damaging agent (e.g., etoposide) to assess the pluripotency of a population of stem cells (e.g., a population of human induced pluripotent stem cells).

Effective techniques for determining whether or not a given induced pluripotent stem cell clone is suitable for clinical grade applications are needed. For example, a scalable, functional assay for defining the quality of human induced pluripotent stem cells is needed to validate release criteria for emerging clinical cell therapy applications. As described herein, DNA damaging agents such as etoposide can be used to determine the pluripotency of induced pluripotent stem cell populations. For example, a sample of cells to be assessed can be collected and exposed to one or more DNA damaging agents. If the cell population has a high level of pluripotency, then most, if not all, the cells will undergo apoptosis in response to exposure to the DNA damaging agent(s). If the cell population has a lower level of pluripotency, then fewer cells will undergo apoptosis in response to exposure to the DNA damaging agent(s). In some cases, measuring the concentration of a DNA damaging agent (e.g., etoposide) needed to induce apoptosis of 50 percent of the cells (an EC₅₀ value) can be used to determine the pluripotency of the cell population. In such cases, an EC₅₀ value for etoposide less than 300 nM can indicate that the cell population has an acceptable level of pluripotency, while an EC₅₀ value for etoposide greater than 300 nM can indicate that the cell population has an unacceptable level of pluripotency.

Having the ability to assess the level of pluripotency of cell populations as described herein can allow clinicians and laboratory personnel to provide reproducible readouts of the innate pluripotent ground state of cell populations (e.g., human induced pluripotent stem cell clones) throughout the entire laboratory process and can allow them to ensure the consistency of induced pluripotent stem cell populations based on functional performance of the pluripotent stem cells, thereby significantly improving the clinical diagnostic integrity of downstream clinical assays and the clinical therapeutic integrity of downstream clinical therapeutic applications.

In some cases, the methods and materials provided herein can be used to release a stem cell population for clinical diagnostic or therapeutic use. For example, a cell sample from a stem cell population can be obtained, exposed to a DNA damaging agent (e.g., etoposide), and assessed to determine the EC₅₀ value of the DNA damaging agent. If the EC₅₀ value for etoposide is less than 300 nM, then the stem cell population can be released for clinical diagnostic or therapeutic use. If the EC₅₀ value for etoposide is greater than 300 nM, then the stem cell population can be discarded or used in applications other than clinical diagnostic use or clinical therapeutic use.

In general, one aspect of this document features a method for releasing a stem cell population for clinical diagnostic or therapeutic use. The method comprises, or consist essentially of, (a) obtaining samples of cells from the stem cell population, (b) contacting the samples with different concentrations of a DNA damaging agent, (c) performing fluorescence-activated cell sorting using the samples to determine that the concentration of the DNA damaging agent that results in about 50 percent of the cells within one of the samples to undergo apoptosis is less than about 300 nM, and (d) releasing the stem cell population for clinical diagnostic or therapeutic use. The stem cell population can be a human induced pluripotent stem cell population. The DNA damaging agent can be etoposide.

In another aspect, this document features a method for releasing a stem cell population for clinical diagnostic or therapeutic use. The method comprises, or consists essentially of, (a) contacting samples of cells from the stem cell population with different concentrations of a DNA damaging agent, (b) performing fluorescence-activated cell sorting using the samples to determine that the concentration of the DNA damaging agent that results in about 50 percent of the cells within one of the samples to undergo apoptosis is less than about 300 nM, and (c) releasing the stem cell population for clinical diagnostic or therapeutic use. The stem cell population can be a human induced pluripotent stem cell population. The DNA damaging agent can be etoposide.

In another aspect, this document features a method for assessing the pluripotency of a stem cell population. The method comprises, or consist essentially of, (a) obtaining samples of cells from the stem cell population, (b) contacting the samples with different concentrations of a DNA damaging agent, and (c) performing fluorescence-activated cell sorting using the samples to determine the concentration of the DNA damaging agent that results in about 50 percent of the cells within one of the samples to undergo apoptosis, wherein the stem cell population exhibits acceptable pluripotency when the concentration is less than 300 nM, and wherein the stem cell population exhibits unacceptable pluripotency when the concentration is greater than 300 nM. The stem cell population can be a human induced pluripotent stem cell population. The DNA damaging agent can be etoposide.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIGS. 1A-E. Human induced pluripotent stem cell clones exhibiting the highest degree of etoposide sensitivity also demonstrated good human induced pluripotent stem cell clonal morphology and pluripotent gene expression patterns consistent with pluripotent control cell lines. (A) 4× images of human induced pluripotent stem cell clones. Outline highlights clones (RT4.1, 6.21) displaying acceptable morphology (clean, distinct borders). (B) qEPCR of RNA isolated from five of human induced pluripotent stem cell clones (RT), two control human induced pluripotent stem cell lines (IMR90, iPSf2), and two control ESC lines (H9, H13). Outline highlights the two clones (RT4.1, 6.21) exhibiting pluripotent gene expression patterns most similar to those displayed by control pluripotent cells. (C) RT4.1 and RT6.21 clones successfully formed teratomas in athymic nude mice. (D) Immunofluorescence detection of SSEA-3 and TRA-1-60 expression in human induced pluripotent stem cells. (E) Annexin V/PI staining of human induced pluripotent stem cells treated with or without etoposide for 24 hours and plotted as a percent of DMSO control. Outline highlights two human induced pluripotent stem cell lines (RT4.1, 6.21) that demonstrated the greatest sensitivity to etoposide.

FIGS. 2A-E. Poor human induced pluripotent stem cell clone quality along with cellular differentiation is reflected by reduced or negligible etoposide sensitivity in human induced pluripotent stem cells cultured in feeder free conditions. (A) Poor quality human induced pluripotent stem cells cultured on GelTrex coated plates exhibited reduced sensitivity to etoposide compared to (B) similarly treated human induced pluripotent stem cells displaying good morphology. (C, D) Removal of poor quality clones/cells from unclean (UC) human induced pluripotent stem cell cultures resulted in increased etoposide sensitivity in the cleaned (C) cultures. (E) The sensitivity of human induced pluripotent stem cells to etoposide is progressively lost over the course of cardiac differentiation.

FIGS. 3A-F. Large scale etoposide sensitivity assays, but not SSEA-4/TRA-1-160 expression, revealed a significant change in etoposide-dependent EC₅₀ values. Panel A provides a summary graph incorporating 126 human induced pluripotent stem cell clones treated with etoposide (or DMSO control) for 24 hours. Solid dots represent the median value of the associated treatment. Panel B provides the mean EC₅₀ values calculated for human induced pluripotent stem cell clones regardless of reprogramming strategy (combined, n=112), while panel C separates the results by lentivirus (n=59) or Sendai virus (n=53). Panel D is a representative example of an SSEA-4/TRA-1-60 dot plot including Tra+ SS+ values used to determine the percent of SSEA-4/TRA-1-60 expression. Panel E provides the mean dual (+) SSEA-4/TRA-1-60 value(s) calculated for human induced pluripotent stem cell clones (Combined, n=100), while panel F separates the Substresults by lentivirus (n=57) or Sendai virus (n=43).

FIGS. 4A-C. Principle component analyses (PCA) revealed a positive correlation between etoposide EC₅₀ values and pluripotent gene expression. No correlation was demonstrated between pluripotent gene expression and the percent dual (+) SSEA-4/TRA-1-60 values. Panel A is a heat map of 89 human induced pluripotent stem cell clones analyzed for seven common markers of pluripotency. Lenti and Sendai reprogramming strategy for each clone is indicated by a corresponding orange (Lenti) or blue (Sendai) dot. Panel B is a PCA of etoposide EC₅₀ values from human induced pluripotent stem cell clones created with lentivirus (n=60) or Sendai virus (n=29) compared with Ct values generated from expression of seven pluripotentcy-related genes listed in FIG. 4A. Sphere size is relatively proportional to EC₅₀ values (e.g., large spheres equate to high EC₅₀ values). Panel C is a PCA of SSEA-4/TRA-1-60 values from human induced pluripotent stem cell clones created with lentivirus (n=18) or Sendai virus (n=18) compared with Ct values as described in FIG. 4B. Sphere size is relatively proportional to % SSEA-4/TRA-1-60 values.

FIG. 5. No significant correlation was observed between the percentage of cells positive for SSEA-4/TRA-1-60 and etoposide sensitivity. Data from 53 (lentiviral n=11; Sendai n=42) clones assayed for surface expression of SSEA-4/TRA-1-60 and etoposide sensitivity were examined via scatter plot analysis for any potential correlation. Linear regression analysis determined that no significant correlation exists between the two data sets.

FIG. 6. Human induced pluripotent stem cell clones harboring cytogenetic anomalies did not exhibit a significant difference in etoposide sensitivity compared to clones with normal karyotypes. The etoposide analyses were performed on human induced pluripotent stem cell clones exhibiting a normal (n=18) or abnormal (n=6) karyotype, and the summarized means were plotted as percentage of DMSO control. Two-way ANOVA analysis indicated that no significant difference (p=0.2690) exists between the individual means regardless of karyotype.

### DETAILED DESCRIPTION

This document provides methods and materials for assessing the pluripotency of stem cell populations as well as methods and materials for releasing stem cell populations for clinical diagnostic or therapeutic use. Any appropriate stem cell population can be assessed for pluripotency or an appropriate level of pluripotency or can be assessed for release for clinical diagnostic or therapeutic use as described herein. For example, human stem cell populations, induced pluripotent stem cell populations, human induced stem cell populations, human embryonic stem cells, and human somatic cell nuclear transfer derived pluripotent stem cells can be assessed and/or released for clinical diagnostic or therapeutic use as described herein.

As described herein, one or more DNA damaging agents (e.g., one, two, three, four, or more DNA damaging agents) can be incubated with cell samples obtained from a population of stem cells to determine the level of sensitivity within the population. Those populations having a high level of sensitivity to the DNA damaging agent(s) have a high level of pluripotency, while those having a low level of sensitivity to the DNA damaging agent(s) have a low level of pluripotency. In some cases, the concentration (e.g., EC₅₀ value) of DNA damaging agent needed to induce apoptosis in about 50 percent of the cells within a sample can be used to determine whether or not the population has a high or low level of pluripotency. For example, samples with EC₅₀ values for etoposide that are less than 300 nM can indicate that the stem cell population is highly pluripotent, while samples with EC₅₀ values for etoposide that are greater than 300 nM can indicate that the stem cell population has a low level of pluripotency.

Any appropriate DNA damaging agent or combination of DNA damaging agents can be used to assess pluripotency or to release stem cell populations for clinical diagnostic or therapeutic use. Examples of DNA damaging agents include, without limitation, etoposide, topoisomerase inhibitors, and drugs that lead to DNA breaks. Etoposide is a topoisomerase inhibitor used to treat testicular and lung cancer. As described herein, the sensitivity to etoposide positively correlates with pluripotent gene expression and clone morphology indicative of clean, pluripotent cells, and is progressively lost following cellular differentiation. In some cases, when using etoposide, an EC₅₀ value less than about 300 nM can indicate that the stem cell population is highly pluripotent, while an EC₅₀ value greater than about 300 nM can indicate that the stem cell population has a low level of pluripotency.

An assay for assessing pluripotency or for releasing a stem cell population for clinical diagnostic or therapeutic use can include obtaining a series of cell samples from the population being assessed. Each of these samples can be contacted with an increasing concentration of one or more DNA damaging agent(s). Any appropriate length of time can be used for the incubation period. For example, the cells can be incubated as 37° C. for between about 30 minutes and about 48 hours (e.g., between about 30 minutes and about 36 hours, between about 30 minutes and about 24 hours, or between about 1 hour and about 24 hours). After this contacting step, the cells can be evaluated using, for example, flow cytometry to determine how susceptible the cells are to apoptosis. In some cases, flow cytometry can be carried out as follows. Briefly, pluripotent stem cells can be seeded in plates (e.g., 24 well plates) and treated with etoposide (Sigma-Aldrich) or DMSO for a period of time (e.g., about 24 hours) at, for example, about 50% confluence. Media and cells can be stained with Annexin V FITC/PI and analyzed via flow cytometry (e.g., via a Gallios (Beckman Coulter) flow cytometer). Data analysis can be performed using Kaluza (Beckman Coulter) software and normalized by dividing the triplicate means (viable non-staining cells) by the corresponding DMSO control.

In some cases, EC₅₀ values can be determined. In such cases, EC₅₀ values for different stem cell populations can be compared to each other to determine which population is more or less pluripotent than the other.

If the cells are determined to have a high level of susceptibility, then the stem cell population can be classified or otherwise identified as being highly pluripotent. In such cases, the stem cell population can be released for clinical diagnostic or therapeutic use. If the cells are determined to have a low level of susceptibility, then the stem cell population can be classified or otherwise identified as having a low level of pluripotency. In such cases, the stem cell population can be discarded or used in areas outside the clinical diagnostic or therapeutic areas.

In some cases, the methods provided herein can be completed in less than three hours.

This document also provides kits for assessing pluripotency or for releasing a stem cell population for clinical diagnostic or therapeutic use. For example, one or more DNA damaging agent (e.g., etoposide) and reagents for assessing cell viability or apoptosis (e.g., Annexin V FITC/PI staining) can be combined as an article of manufacture such as a kit. In one embodiment, a kit can contain etoposide and Annexin V FITC/PI for assessing cellular viability and early apoptosis to determine the EC₅₀ value for etoposide. In some cases, a kit can contain buffers, positive control samples, dose response reference ranges to interpret data, or combinations thereof. The reagents within a kit can be housed together in various combinations or can be packaged in separate vials or containers. The kits provided herein also can include labels or packaging inserts setting out instructions for preparation and use.

The invention will be further described in the following examples, which do not limit the scope of the invention described in the claims.

### EXAMPLES

### Example 1

### Etoposide Sensitivity Assay for Assessing Pluripotency in Human Induced Pluripotent Stem Cells

Human pluripotent stem cell clones were assessed for etoposide sensitivity as follows. Mechanically processed hiPSC's were seeded onto a 24-well plate (1×60 mm dish: 1×24-well plate), maintained for 3-5 days, and subsequently treated with etoposide (1, 10, 50, 100, 250, 500, and 1,000 nM, all performed in triplicate wells) or vehicle control (DMSO) for 24 hours. Conditioned media was collected into appropriately labeled 12×75 mm flow tubes, and 200 μL of TryPLE was added to each well. Following a brief incubation at 37° C. (5-10 minutes), 1 mL of pre-warmed (37° C.) FAC's buffer (4 mM EDTA, 0.5% FBS, 1×DPBS pH 7.4 w/o Ca²⁺, Mg²⁺) was added to each well. The total well contents were transferred to their corresponding conditioned media tubes, and all tubes were spun down at 400×G for 5 minutes. Tubes were washed 1× with ice-cold Annexin Binding Buffer (ABB, 10 mM HEPES, 2.5 mM CaCl₂, 140 mM NaCl, pH 7.4) and stained with Annexin-FITC (BD 556420, 5 μL in 100 μL total ABB per tube) for 30 minutes on ice while protected from light. All tubes were washed 1× in ABB, and the resulting cell pellets were resuspended in 400 μL of ABB supplemented with 1 μL of 1 mg/mL propidium iodide (Sigma-Aldrich, P4864). Samples were immediately assayed by flow cytometry (Gallios, Beckman Coulter), and data were subsequently analyzed for percent viability using Kaluza software (Beckman Coulter).

These human induced pluripotent stem cell clones also were assessed morphologically by phase contrast light microscopy. In addition, quantitative rtPCR was performed to assess gene expression by the human induced pluripotent stem cell clones. Briefly, expression of REX01, SALL4, TDGF1, SOX2, POU5F1, C-MYC, and ZFP42 were used to distinguish pluripotent stem cells compared to non-stem cell fibroblast lines. Additionally, Tra-1-60 and SSEA-4 cell surface markers were used to quantitate percentage of cell population that expressed high levels of pluripotent cell surface markers. The gene expression and cell surface expression was used as the gold-standard definition of pluripotent stem cells.

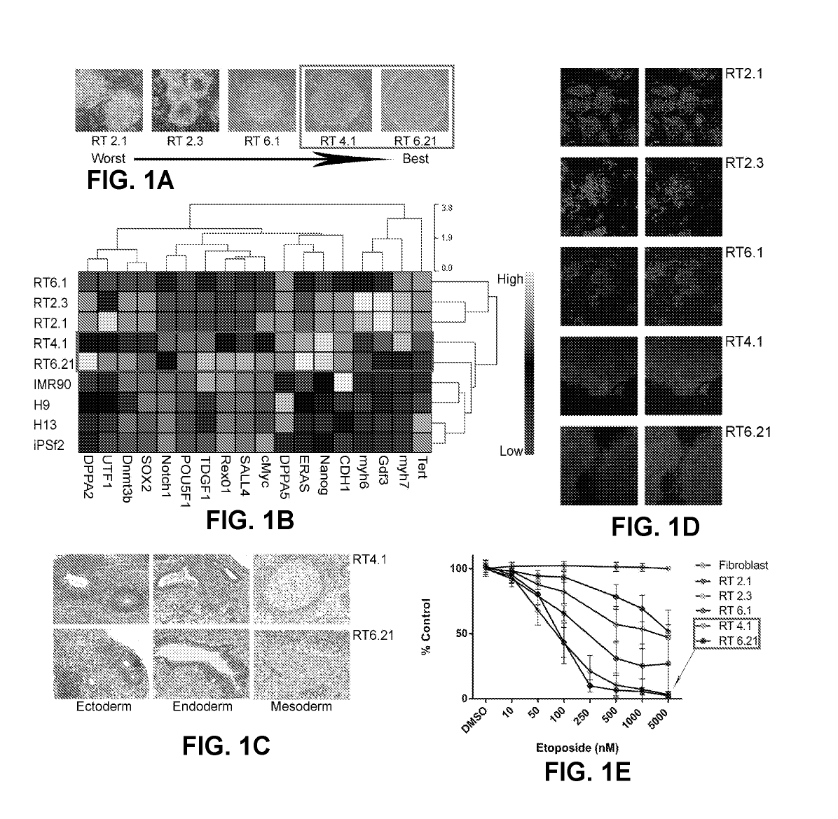

Human pluripotent stem cell clones exhibiting the highest degree of etoposide sensitivity (FIG. 1E) also demonstrated good human induced pluripotent stem cell clonal morphology and pluripotent gene expression patterns consistent with pluripotent control cell lines (FIGS. 1A and 1B). These results demonstrate that DNA damage response is a quantitative readout for the qualitative endpoint of cellular morphology.

No clear difference in SSEA-3/TRA-1-60 expression was observed in any of the hiPSC lines tested (FIG. 1D). However, the lines displaying the best morphology, RNA expression patterns, and greatest sensitivity to etoposide (RT 4.1 and RT 6.21) successfully formed teratomas comprised of all three primary germ layer tissue types (FIG. 1C). These results demonstrate that characterization by ESA corroborates results obtained by teratoma formation, which is the current gold standard for determining pluripotent potential in hiPSC's.

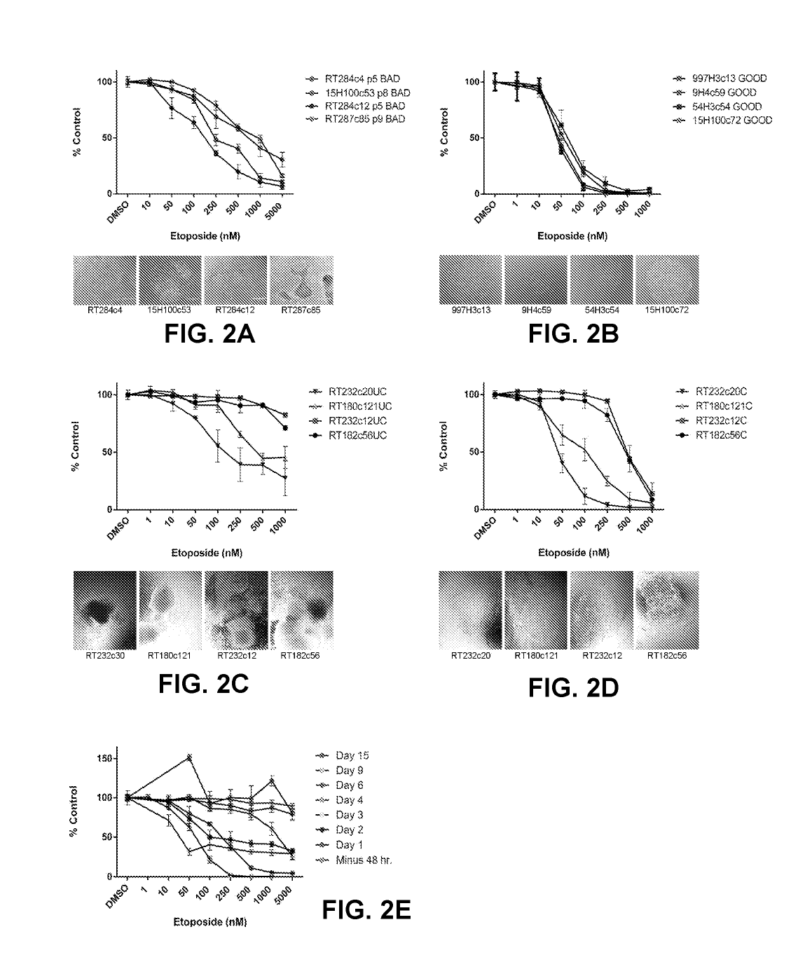

The results provided herein demonstrate that human pluripotent stem cell clones exhibiting poor quality and cellular differentiation exhibited reduced or negligible etoposide sensitivity when cultured in feeder free conditions (FIGS. 2A and 2B). In another experiment, poor quality clones/cells from unclean (UC) human induced pluripotent stem cell cultures were quantitatively identified with greater than 300 nM EC₅₀. This removal of poor quality clones/cells from UC human induced pluripotent stem cell cultures resulted in increased etoposide sensitivity (EC₅₀ below 300 nM) in the cleaned (C) cultures (FIGS. 2C and 2D). These results demonstrate heterogeneous pluripotent stem cell cultures can be manually cleaned to improve the quality of overall population of cells and decrease the EC₅₀ value of the population.

In another experiment, hiPSC's were differentiated into cardiomyocytes using the PSdif-Cardio kit (StemRD). The results revealed a near stepwise decrease in sensitivity toward etoposide throughout the progression of cardiomyocyte differentiation (FIG. 2E). Significantly, mature, beating cardiomyocytes (Day 9) displayed a near complete lack of sensitivity to etoposide, which mimics results obtained from etoposide treated human fibroblasts. This observation demonstrates that not only can an etoposide sensitivity assay (ESA) be used to determine hiPSC pluripotency, but it also provides a means for pre-screening long-term differentiation cultures several days prior to being able to observe gross morphological indices including beating.

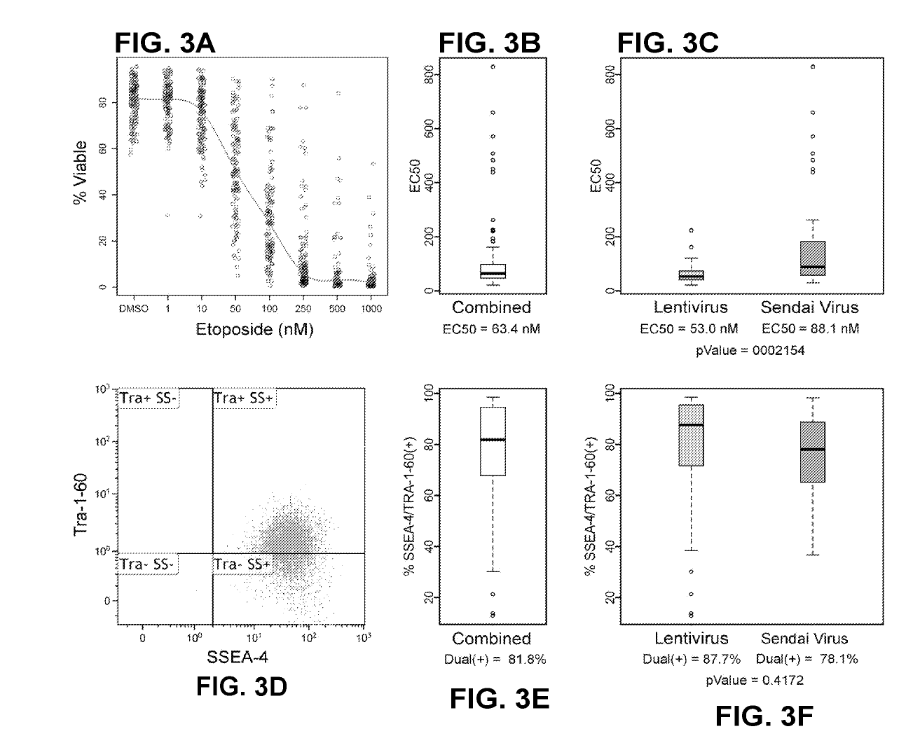

The hiPSC lines were originally generated from dermal fibroblasts using lentivirus delivered reprogramming factors. Later, a Sendai virus reprogramming system was used in order to minimize viral DNA insertions. ESA conducted on 126 hiPSC clones (FIGS. 3A and 3B) demonstrated that Sendai virus derived hiPSC clones exhibited a significant decrease in etoposide sensitivity compared to clones originating from lentivirus reprogramming (FIG. 3C).

Flow-based detection of SSEA-4 and TRA-1-60 (FIG. 3D) in 112 hiPSC clones revealed detectable levels of both cell surface markers in nearly 82% of the counted events (FIG. 3E). However, reprogramming strategy did not appear to significantly affect cell surface expression of either epitope (FIG. 3F). These results demonstrate that an ESA can have a remarkable degree of consistency when applied to large numbers of hiPSC's, and that an ESA can detect and quantify relatively small changes in pluripotency.

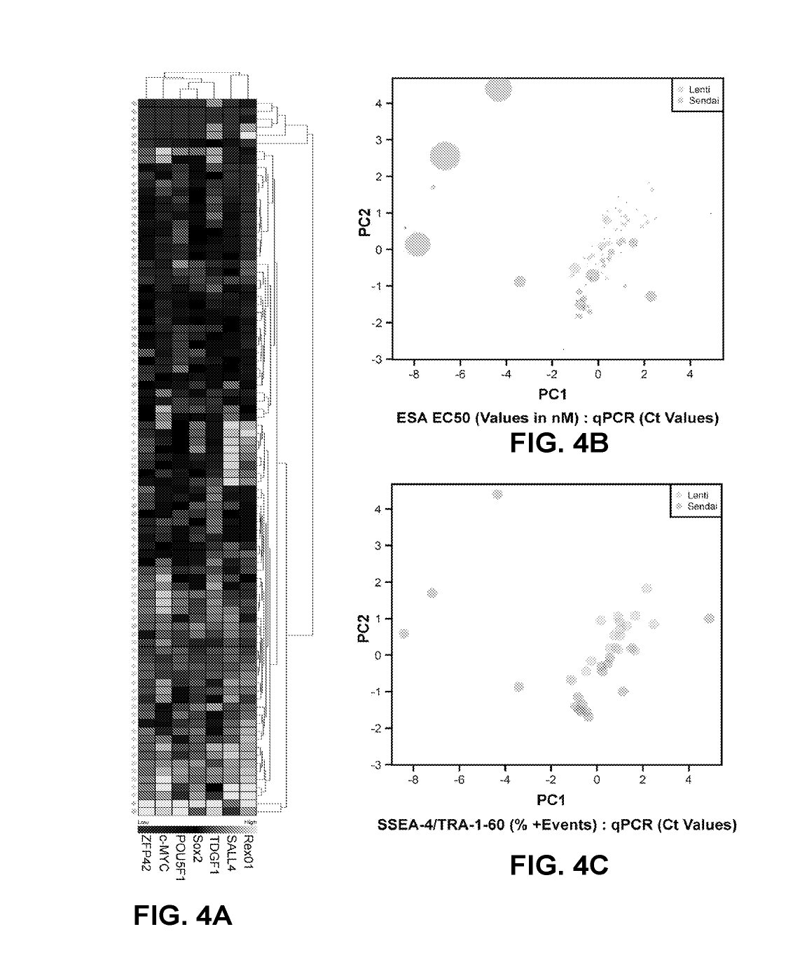

RNA expression is often employed as a high throughput means of assessing hiPSC pluripotency. Results from the ESA were compared to qPCR data obtained from a subset of clones to determine if a statistically positive correlation exists. qPCR analyses were performed on 89 hiPSC clones and delineated by expression level via heat map projection (FIG. 4A). Principle component analysis demonstrated a positive correlation between hiPSC clones exhibiting a low ESA EC₅₀ value (small diameter globes) and RNA expression patterns associated with pluripotency (lower, left-hand cluster) (FIG. 4B). Conversely, SSEA-4/TRA-1-60 co-expression (greater expression=larger globe) was not significantly correlated with pluripotent RNA expression patterns (FIG. 4C). These results demonstrate that ESA provides a similar degree of reliability in predicting hiPSC pluripotency on large numbers of hiPSC clones commonly associated with RNA expression analyses.

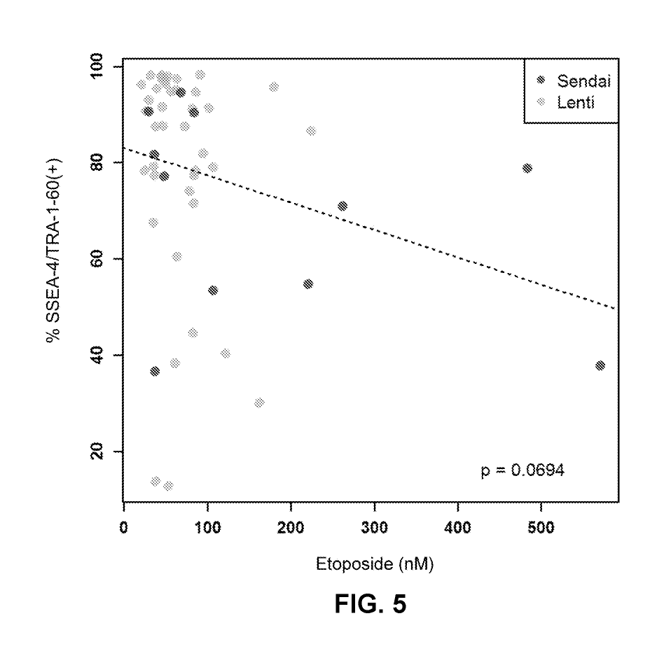

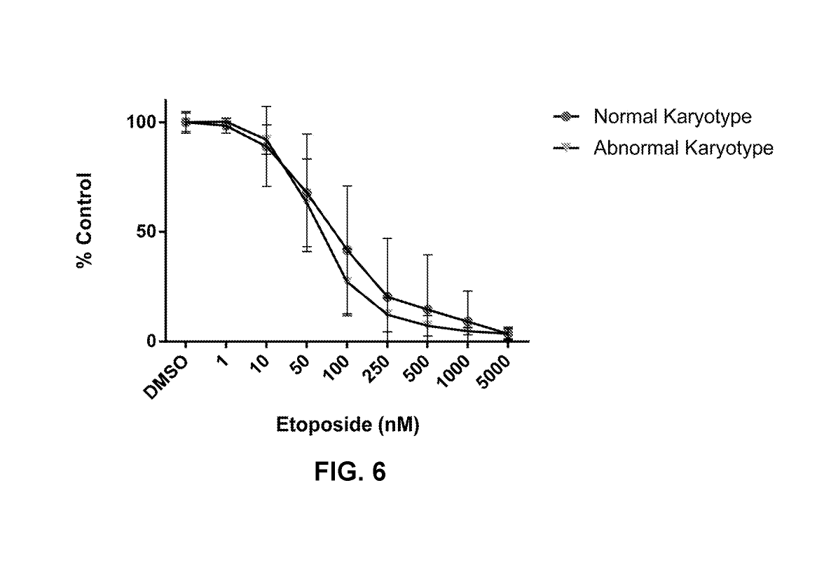

Flow-based % SSEA-4/TRA-1-60 cell surface expression was compared to ESA EC₅₀ values obtained from 52 hiPSC clones. Higher levels of SSEA-4/TRA-1-60 expression trended with but were not significantly correlated to increased etoposide sensitivity (FIG. 5). Additionally, karyotypic abnormalities determined in 6 hiPSC lines (Table 1) did not affect ESA results when compared to hiPSC clones (n=18) exhibiting a normal karyotype (FIG. 6). These results demonstrate that elevated sensitivity to etoposide is not always associated with increased % SSEA-4/TRA-1-60 cell surface expression on hiPSC's, a result that combined with that summarized in FIG. 4C, demonstrates the enhanced ability ESA maintains in determining hiPSC pluripotency compared with that exhibited by quantification of SSEA-4 and TRA-1-60 expression. In addition, ESA EC₅₀ values were not significantly impacted by hiPSC's harboring an abnormal karyotype, at least in the current study.

TABLE 1
Summary of human pluripotent stem cell cytogenetic analysis.

| hiPS I.D. | Cytogenetic Abnormality | Abnormal Frequency |
|---|---|---|
| 15H1c2 | Supernumerary Ring Chromosome | 2/20 Metaphases |
| | of Unknown Origin | |
| 9H1c3 | Trisomy 12 | 12/28 Metaphases |
| 6c163 | Trisomy 12 | 14/20 Metaphases |
| | 46, XX, +12, −21 | 1/20 |
| | 46, XX, +12, −18 | 1/20 |
| 13c54 | 47, XX, +X | 2/20 Metaphases |
| | 45, X, −X | 1/20 |
| 3c59 | Trisomy 12 | 13/20 Metaphases |
| 3c61 | Trisomy 12 | 12/20 Metaphases |

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for releasing a stem cell population for clinical diagnostic or therapeutic use, wherein said method comprises:
(a) obtaining samples of cells from said stem cell population,
(b) contacting said samples with different concentrations of a DNA damaging agent,
(c) performing fluorescence-activated cell sorting using said samples to determine that the concentration of said DNA damaging agent that results in about 50 percent of the cells within one of said samples to undergo apoptosis is less than about 300 nM, and
(d) releasing said stem cell population for clinical diagnostic or therapeutic use.

2. The method of claim 1, wherein said stem cell population is a human induced pluripotent stem cell population.

3. The method of claim 1, wherein said DNA damaging agent is etoposide.

4. A method for releasing a stem cell population for clinical diagnostic or therapeutic use, wherein said method comprises:
(a) contacting samples of cells from said stem cell population with different concentrations of a DNA damaging agent,
(b) performing fluorescence-activated cell sorting using said samples to determine that the concentration of said DNA damaging agent that results in about 50 percent of the cells within one of said samples to undergo apoptosis is less than about 300 nM, and
(c) releasing said stem cell population for clinical diagnostic or therapeutic use.

5. The method of claim 4, wherein said stem cell population is a human induced pluripotent stem cell population.

6. The method of claim 4, wherein said DNA damaging agent is etoposide.

7. A method for assessing the pluripotency of a stem cell population, wherein said method comprises:
(a) obtaining samples of cells from said stem cell population,
(b) contacting said samples with different concentrations of a DNA damaging agent, and
(c) performing fluorescence-activated cell sorting using said samples to determine the concentration of said DNA damaging agent that results in about 50 percent of the cells within one of said samples to undergo apoptosis, wherein said stem cell population exhibits acceptable pluripotency when said concentration is less than 300 nM, and wherein said stem cell population exhibits unacceptable pluripotency when said concentration is greater than 300 nM.

8. The method of claim 7, wherein said stem cell population is a human induced pluripotent stem cell population.

9. The method of claim 7, wherein said DNA damaging agent is etoposide.
